(19) United States
(12) Patent Application Publication
(10) Pub. No.: US 2021/0346537 A1
(43) Pub. Date: Nov. 11, 2021

(54) SANITIZER FOR COSMETICS

(71) Applicant: Stella Song, La Habra Heights, CA (US)

(72) Inventor: Stella Song, La Habra Heights, CA (US)

(73) Assignee: Stella Song

(21) Appl. No.: 16/869,395

(22) Filed: May 7, 2020

(51) Int. Cl. A61L 2/10; A61L 2/26; A45D 40/20

(52) U.S. Cl. CPC A61L 2/10; A61L 2/26; A45D 40/20; A61L 2202/10; A45D 2200/205; A61L 2202/20; A45D 2200/10

(57) ABSTRACT

An applicator wand for the cosmetic product is optically coupled to an ultraviolet light source as an illumination source of ultraviolet light wavelengths. The applicator wand provides the source of the ultraviolet light wavelengths to be a germicide for bacteria in the cosmetic product on the applicator wand. The ultraviolet light source emits the ultraviolet light wavelengths in a range extending between 202-225 nm. A container contains the cosmetic product. The cap of the container attaches to a body of the container. One or more batteries electrically connect to at least an electronic control circuit and the ultraviolet light source.

### NOTICE OF COPYRIGHT

A portion of this disclosure contains material that is subject to copyright protection. The copyright owner has no objection to the facsimile reproduction by anyone of the material subject to copyright protection as it appears in the United States Patent & Trademark Office's patent file or records, but otherwise reserves all copyright rights whatsoever.

### FIELD

Embodiments of the design provided herein generally relate to a sanitizer for cosmetics.

### BACKGROUND

Ultraviolet-C (UVC) light is generally electromagnetic irradiation at wavelengths of 200 to 280 nm.

### SUMMARY

In an embodiment, a system, a method, and an apparatus are discussed for a sanitizer for cosmetics. The applicator wand for the cosmetic product is optically coupled to an ultraviolet light source as an illumination source of ultraviolet light wavelengths. The applicator wand provides the source of the ultraviolet light wavelengths to be a germicide for bacteria in the cosmetic product on the applicator wand. The ultraviolet light source emits the ultraviolet light wavelengths in a range extending between 202-225 nm. A container contains the cosmetic product. The cap of the container attaches to a body of the container. One or more batteries electrically connect to at least an electronic control circuit and the ultraviolet light source.

This and many more example embodiments are discussed herein.

### BRIEF DESCRIPTION OF THE DRAWINGS

FIG. 1 illustrates a block diagram of an embodiment of an applicator wand for the cosmetic product that is optically coupled to an ultraviolet light source as an illumination source of ultraviolet light wavelengths.

FIG. 2 illustrates a block diagram of an embodiment of the applicator wand has an outer body with an opening at a bottom of the outer body to the cosmetic product in the container, where the outer body is attached to a suction head to function as a syringe and dropper for a fluid-based cosmetic product.

FIG. 3 illustrates a block diagram of an embodiment of the container has the one or more additional ultraviolet light sources built into an interior of the container, where the container has i) a tubular or ii) rectangular tube-shape, where the one or more additional ultraviolet light sources are configured in signal strength and choice of emitted ultraviolet wavelengths to act as another germicide for the cosmetic product contained in the container itself in addition to cooperating with one or more ultraviolet light sources emitting ultraviolet light wavelengths from the applicator wand to act as a germicide for the cosmetic product on the applicator wand itself.

FIG. 4 illustrates a block diagram of an embodiment of two or more additional ultraviolet light sources are built into walls of the container where the two or more additional ultraviolet light sources are arranged in a pattern inside the container.

While the design is subject to various modifications, equivalents, and alternative forms, specific embodiments thereof have been shown by way of example in the drawings and will now be described in detail. It should be understood that the design is not limited to the particular embodiments disclosed, but—on the contrary—the intention is to cover all modifications, equivalents, and alternative forms using the specific embodiments.

### DETAILED DESCRIPTION

In the following description, numerous specific details can be set forth, such as examples of specific data signals, named components, types of cosmetics, etc., in order to provide a thorough understanding of the present design. It will be apparent, however, to one of ordinary skill in the art that the present design can be practiced without these specific details. In other instances, well known components or methods have not been described in detail but rather in a block diagram in order to avoid unnecessarily obscuring the present design. Further, specific numeric references such as the first light source, can be made. However, the specific numeric reference should not be interpreted as a literal sequential order but rather interpreted that the first light source is different than a second light source. Thus, the specific details set forth can be merely exemplary. This disclosure describes inventive concepts with reference to specific examples. However, the intent is to cover all modifications, equivalents, and alternatives of the inventive concepts that are consistent with this disclosure. It will be apparent, however, to one of ordinary skill in the art that the present approach can be practiced without these specific details. Thus, the specific details set forth are merely exemplary, and is not intended to limit what is presently disclosed. The specific details can be varied from and still be contemplated to be within the spirit and scope of the present design. The term “coupled” is defined as meaning connected either directly to the component or indirectly to the component through another component. The features implemented in one embodiment may be implemented in another embodiment where logically possible.

Provided herein is a sanitizer for cosmetics including in an embodiment, a cap, an applicator wand as an illumination source of ultraviolet light wavelengths, a tubular or a rectangular tube-shaped container to contain a cosmetic product, one or more batteries, one or more illumination lights to illuminate an area where the cosmetic product is intended to be applied, and other portions, including an electronic control circuit, of a cosmetic sanitizing system. The applicator wand provides the source of the ultraviolet rays to be germicidal for bacteria on the applicator wand as well as in the cosmetic product contained in the container itself.

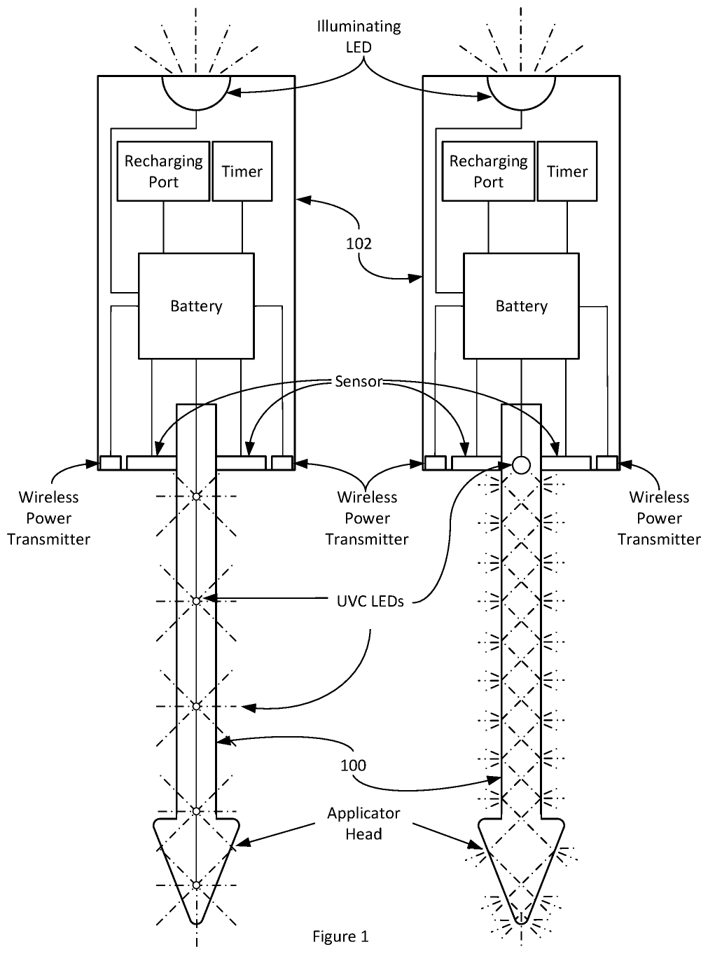

FIG. 1 illustrates a block diagram of an embodiment of an applicator wand 100 for the cosmetic product that is optically coupled to an ultraviolet light source as an illumination source of ultraviolet light wavelengths.

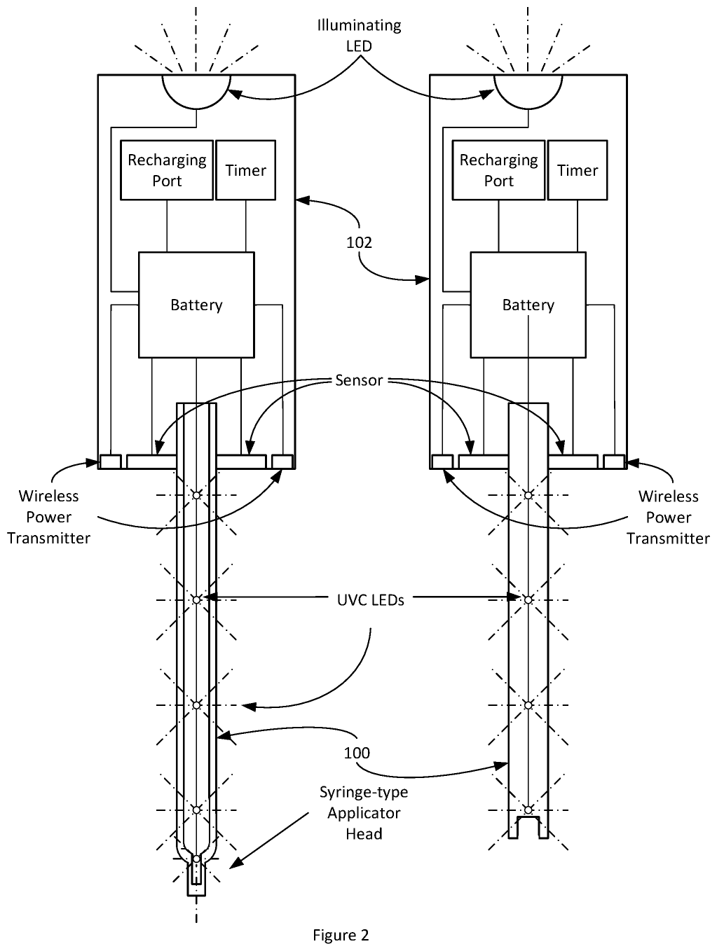

FIG. 2 illustrates a block diagram of an embodiment of the applicator wand 100 has an outer body with an opening at a bottom the outer body to the cosmetic product in the container 104, where the outer body is attached to a suction head to function as a syringe and dropper for a fluid-based cosmetic product.

### Applicator Wand

Referring to FIGS. 1 and 2, the applicator wand 100 is connected into the cap 102. The applicator wand 100 is a translucent structure made partially or entirely from glass, in order to keep a more sterile surface. The glass can help to limit an amount of cosmetic product, such as lip gloss, mascara, etc., that builds up on the body of the applicator wand 100 as well as on the removable head. In general, the one or more ultraviolet light sources are located in the cap 102 and optically couple to a body and head of the applicator wand 100. The body of the glass applicator wand 100 may be translucent to allow the ultraviolet light from the ultraviolet light sources, such as one or more light emitting diodes, in the cap 102 to route the light down and out of the body and head of the glass applicator wand 100. In an embodiment, the light emitting diodes couple to a top of the applicator wand. In an embodiment, an ultraviolet light source in the cap couples to multiple thin fiber-optic tubes that run down the length of the glass applicator wand 100 to carry and emit the ultraviolet light wavelengths throughout the entire length of the applicator wand 100. In an embodiment, multiple LEDs optically run down a length of the body of the applicator wand. The multiple fiber optic tubes couple to the ultraviolet light source in the cap 102. One or more optical filters can optically couple between these two components to control the range of ultraviolet light wavelengths being transmitted and emitted throughout the entire length of the applicator wand 100. This wavelength range of ultraviolet light can be narrowed to a single wavelength, such as 222 nm.

A head of the applicator wand 100 connects to a body of the applicator wand 100. The applicator head may be bristleless or may have bristles that are easily cleanable. The applicator head may be removable and replaceable from the body of the applicator wand 100. The applicator head may, for example, i) screw onto and off of, ii) mechanically snap onto and off of, the body of the applicator wand 100. The applicator head generally may have a transparent interior structure. The applicator wand 100 may be designed to be reusable by having the ability to attach to a cap 102 of containers 104 for multiple different container 104s, such as containers 104 from different brands of cosmetics.

Referring to FIG. 2, the applicator wand 100 may be a dropper with a glass-based tubular inner portion to contain the ultraviolet-C LEDs and a syringe-like outer portion in order to provide suction for fluids, e.g. cosmetic serums, while maintaining the ability to emit the ultraviolet light wavelengths from within the glass-based tubular inner portion of the applicator wand 100. The applicator wand 100 has both

i) an outer body with an opening at a bottom of the outer body to the cosmetic product in the container 104. The outer body is attached to a suction head to function as a syringe and dropper for a fluid-based cosmetic product, and

ii) a glass-based tubular inner portion that is optically coupled to the ultraviolet light source as the illumination source of the ultraviolet light wavelengths to emit the ultraviolet light wavelengths from within the glass-based tubular inner portion of the applicator wand 100.

### Batteries and Charging Circuit

Referring to FIGS. 1 and 2, the one or more batteries are configured to be located in and embedded inside the cap 102. The batteries may be rechargeable batteries, such as lithium-ion batteries. The batteries are configured to power the cosmetic sanitizing system including the one or more illumination lights, the ultraviolet light source, and the control circuitry. The one or more rechargeable batteries connect to a recharging circuit which includes a USB port, USB-C port, micro-USB, mini-USB port, etc. formed in the cap 102 in order to recharge the batteries.

### Sanitizing Process and Control System

Referring to FIGS. 1 and 2, the cosmetic sanitizer system may have one or more ultraviolet light sources built into i) the applicator wand 100, ii) the tubular or rectangular tube-shaped container 104, or iii) a combination thereof. The cosmetic sanitizer system may include a control circuit including a timer, a wireless power transmitter, a wireless power receiver, and one or more sensors. In an embodiment, the ultraviolet light source may be diode-based (e.g. LEDs), Cold Cathode, Excimer Lamp such as a krypton-chlorine (Kr—Cl) excimer, or another ultraviolet light source. The ultraviolet light source may be configured and designed to minimize heat generation and/or direct heat away from the head of the applicator wand 100. This ensures several benefits. The heat is substantially kept away from the user's skin, lips and eyes if the wand application is used by the person recently after a sanitizing session occurs. The heat is substantially kept away from the cosmetic product itself, which helps prevent drying and build-up of the cosmetic product as well as any altering of the cosmetic product due to heat.

The ultraviolet light source in the cosmetic sanitizer system can emit a light in the Far Ultraviolet C (“ultraviolet-C”) range extending between (202-225 nm) from one or more lights. The control circuit with a timer is configured to ensure the cosmetic product housed within the container 104 is exposed to the ultraviolet light wavelengths for a fixed amount of time during a sanitizing session. For example, the timer may have the ultraviolet light source come on for a time period of ten minutes to sanitize and kill germs (bacteria or viruses) that are in the cosmetic product and/or on the applicator wand 100. The electronic control circuit uses one or more sensors to detect when the cap 102 is secured onto the container 104 before activating the one or more ultraviolet light sources to emit the ultraviolet light wavelengths merely when the applicator wand 100 is inside the container 104, which is indicated by the cap 102 being secured onto the container 104. The one or more sensors, as a safety measure, are configured to detect when the cap 102 is closed onto the container 104 before activating the cosmetic sanitizer system to avoid directly exposing the ultraviolet-C light to human skin or eyes. For an example sensor, a normally electrically open contact can be used such that when the cap 102 is off the container 104, then the electrically open contact remains open, which shuts off the power to the ultraviolet light source. When the cap 102 is on the container 104, then the electrically open contact goes closed allowing power to reach the ultraviolet light source. Note, the timer may also be programmable to run once every fixed period of time after an initial sanitizing session occurs. For example, a cosmetic product may be applied, the cap 102 and the attached wand application secured back onto the container 104, which will trigger the sanitizing session when the cap 102 is put back on the container 104. However, this cosmetic applicator and product may then sit unused for several days. The programmable time period can be programmed to run once every fixed period of time after the initial sanitizing session. For example, a subsequent sanitizing session may be run at least once every 48 hour period even if the cap 102 has not been removed from the container 104 in order to act as a germicide for the applicator including its head and the cosmetic product in the container 104.

Another mechanism to keep the cosmetic product bacteria free is use of a gasket. The cap 102 to container 104 connection may have a rubber gasket positioned at that location. The applicator wand 100 extending out of the cap 102 can penetrate an air tight seal in the gasket to extend into the body of the container 104 where the cosmetic product is stored. The air tight seal in the gasket in a neck of the container 104 minimizes an amount the cosmetic product is exposed to air, which aids in minimizing keeping the cosmetic product on the applicator wand 100 free from harmful bacteria.

Another mechanism to aid in minimizing keeping the cosmetic product on the applicator wand 100 free from harmful bacteria is the applicator wand 100 itself. The applicator wand 100 itself provides the source of the ultraviolet rays to be germicidal for bacteria on the applicator wand 100. The germicidal mechanism of the ultraviolet light relies on its DNA-damaging effect on bacteria. Irradiation with ultraviolet light waves from 204 nm ultraviolet light up to 225 nm ultraviolet light significantly reduce bacterial numbers on the applicator wand 100 and in the cosmetic product itself.

The ultraviolet light source may emit an ultraviolet-C light at, for example, 222 nm. Ultraviolet-C light at large doses of 222 nm can pose no substantial potential health risks to the skin or eyes. The 222 nm ultraviolet light can be more effective than conventional ultraviolet sources at certain types of bacteria eradication and, for example, is more effective at eradicating most bacterial spores than, for example, 254 nm light. In an embodiment, a range of ultraviolet light waves may be used from 200 nm ultraviolet light up to 225 nm ultraviolet light.

In general, the electronic control circuit is configured such that ultraviolet light will be emitted when the applicator wand 100 is inside the container 104 and the timer is periodically running the sanitizing cycle. In general, the electronic control circuit is configured such that ultraviolet light will be off and not be emitted from the applicator wand 100 when outside of the container 104. However, ultraviolet light waves from, for example, 204 nm ultraviolet light up to 225 nm ultraviolet light have been chosen because they are effective at being germicidal and in general do not substantially harm human cells. In particular, irradiation with a 222 nm may be an optimum combination of effective disinfection and biological safety to human cells. Again, one or more optical filters or other techniques may be used to ensure the applicator wand 100 emits a single wavelength, such as 222 nm.

The cosmetic sanitizer has been made safe to be carried in a purse or handbag all day by a human and not be a danger to that person. Again, the container 104 is designed to contain the ultraviolet wavelengths within the container 104 or at least reduce the strength of the ultraviolet wavelengths going outside of the container 104 during a sanitizing session. Direct exposure from the ultraviolet light waves from 204 nm ultraviolet light up to 225 nm ultraviolet light have been shown to absorbed by proteins in the membrane and cytosol, but fail to reach the nucleus of human cells. In addition, the cosmetic sanitizer has been made safe in case a fault occurs and the ultraviolet light accidently comes on when near the person when they are attempting to apply the cosmetics.

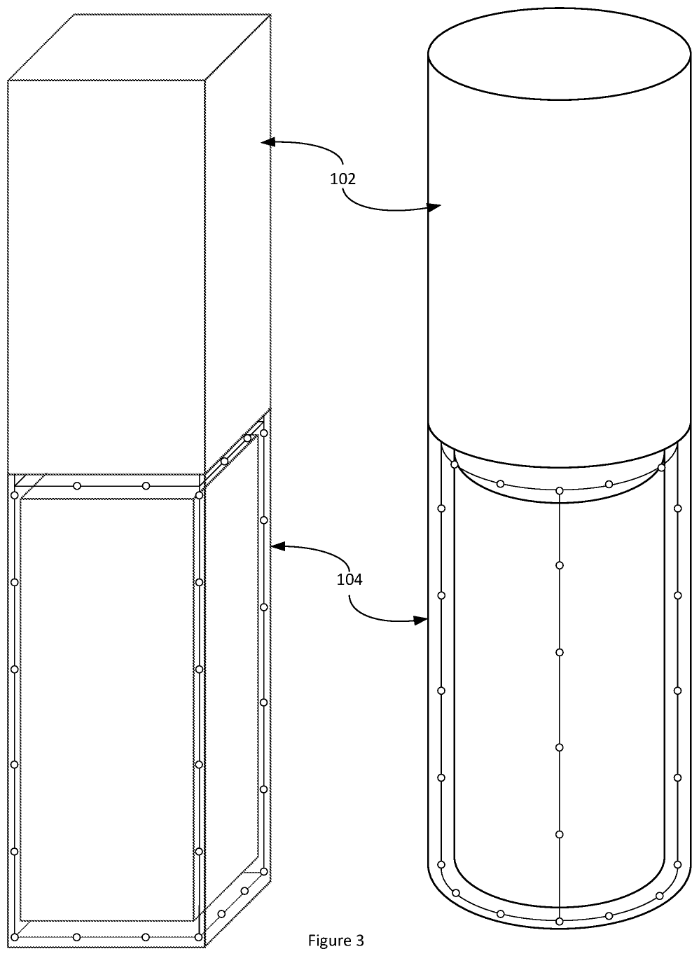

FIG. 3 illustrates a block diagram of an embodiment of the container 104 that has the one or more additional ultraviolet light sources built into an interior of the container 104. The container 104 can have i) a tubular shape or ii) rectangular tube-shape. The one or more additional ultraviolet light sources are configured in signal strength and choice of emitted ultraviolet wavelengths, such as 222 nm or 204 nm, to act as another germicide for the cosmetic product contained in the container 104 itself in addition to cooperating with one or more ultraviolet light sources emitting ultraviolet light wavelengths from the applicator wand 100 to act as a germicide for the cosmetic product on the applicator wand 100 itself.

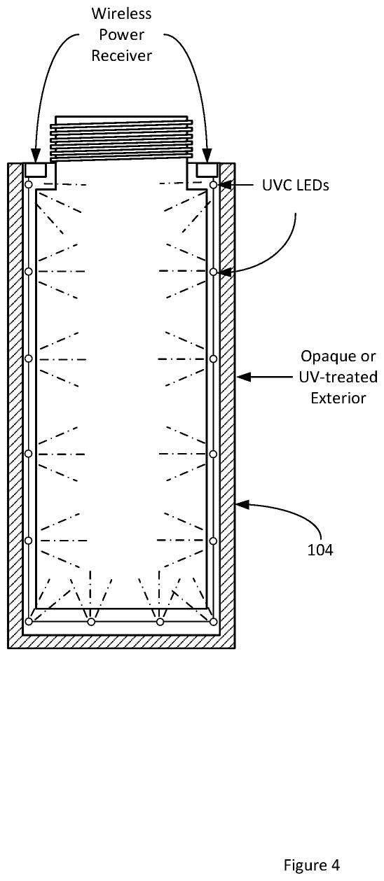

FIG. 4 illustrates a block diagram of an embodiment of two or more additional ultraviolet light sources that are built into walls of the container 104 where the two or more additional ultraviolet light sources are arranged in a pattern inside the container 104.

Referring to FIGS. 3 and 4, the container 104 of the cosmetic sanitizer system may have one or more ultraviolet light sources built into interior of the tubular or rectangular tube-shaped container 104.

### Illumination Light

Referring to FIGS. 1 and 2, one or more illumination lights attach to the cap 102 and/or the applicator wand 100 to illuminate an area where the cosmetic product is intended to be applied. The one or more illumination lights are electrically connected to the one or more batteries, and are physically attached to i) the cap 102, ii) the applicator wand 100, iii) the container 104, and iv) any combination of these. The one or more illumination lights may be comprised of light-emitting diodes (“LEDs”) configured to emit light in the range of wavelengths within the visible spectrum (380-740 nm). The one or more lights may be embedded in several locations along the cosmetic item for aesthetic purposes, e.g. on the cap 102, around the container 104, etc.

### Cap and Container

Referring to FIGS. 3 and 4, the cap 102 of a container 104, such as a lid, can be attached to a body of the container 104, such as a bottle, by either screwing the cap 102 onto a threaded neck of the container 104, the cap 102 snapping with a locking mechanism onto a top of the container 104, or a similar mechanism. The cap 102 can house the electronic control circuit, which can engage a mechanical locking mechanism between the cap 102 and the body of the container 104 for a duration of time the ultraviolet light source is actively emitting the ultraviolet light wavelengths. The cap 102 may have a cooling system, such as a vent. The container 104 may have a darkened or ultraviolet-treated exterior to limit exposure of the ultraviolet-C light wavelengths to an outside environment.

The applicator wand 100 may be designed to be reusable by having the ability to attach to a cap 102 of container 104s for multiple different container 104s, such as container 104s from other brands and other cosmetic products, and the selected range of ultraviolet wavelengths allow a same instance of the applicator wand 100 to be used as a germicide for multiple different types of cosmetic products. Thus, the different container 104s each containing one of the plurality cosmetic products that the same applicator wand 100 can be used to sanitize their cosmetic products.

While the foregoing design and embodiments thereof have been provided in considerable detail, it is not the intention of the applicant(s) for the design and embodiments provided herein to be limiting. Additional adaptations and/or modifications are possible, and, in broader aspects, these adaptations and/or modifications are also encompassed. Accordingly, departures may be made from the foregoing design and embodiments without departing from the scope afforded by the following claims, which scope is only limited by the claims when appropriately construed.

1. A sanitizer for cosmetic product, comprising:
a cap;
an applicator wand for the cosmetic product that is optically coupled to an ultraviolet light source as an illumination source of ultraviolet light wavelengths, where the applicator wand is configured to provide the source of the ultraviolet light wavelengths to be a germicide for bacteria in the cosmetic product on the applicator wand, where the ultraviolet light source is configured to emit the ultraviolet light wavelengths in a range extending between 202-225 nm;
a container is configured to contain the cosmetic product, where the cap of the container is configured to be attached to a body of the container; and
one or more batteries electrically connected to an electronic control circuit and the ultraviolet light source.

2. The apparatus of claim 1, further comprising:
one or more illumination lights configured to illuminate an area where the cosmetic product is intended to be applied, where the one or more illumination lights are electrically connected to the one or more batteries, and are physically attached to i) the cap, ii) the applicator wand, iii) the container, and iv) any combination of these, where the one or more illumination lights are configured to emit light in a range of wavelengths within a visible spectrum (380-740 nm).

3. The apparatus of claim 2, where the cap is configured to house the electronic control circuit, which is configured to engage a mechanical locking mechanism between the cap and the body of the container for a duration of time the ultraviolet light source is actively emitting the ultraviolet light wavelengths.

4. The apparatus of claim 1, where the one or more batteries are located in and embedded inside the cap, where the batteries are rechargeable batteries and connect to a recharging circuit which includes any of i) a USB port, ii) USB-C port, iii) micro-USB, and iv) mini-USB port, formed in the cap in order to recharge the rechargeable batteries.

5. The apparatus of claim 1, where the applicator wand is connected into the cap, where the applicator wand is a translucent structure made partially or entirely from glass, where the one or more ultraviolet light sources are located in the cap and optically couple to a body and head of the applicator wand.

6. The apparatus of claim 1, where a head of the applicator wand is configured to connect to a body of the applicator wand, where the applicator head is configured to be removable and replaceable from the body of the applicator wand.

7. The apparatus of claim 1, where the one or more ultraviolet light sources are built into the cap and then optically coupled to the applicator wand; and then, two or more additional ultraviolet light sources are built into walls of the container where the two or more additional ultraviolet light sources are arranged in a pattern inside the container, and where the one or more ultraviolet light sources are configured to emit the ultraviolet light wavelengths at 222 nm.

8. The apparatus of claim 7, where the container has the one or more additional ultraviolet light sources built into an interior of the container, where the container has i) a tubular or ii) rectangular tube-shape, where the one or more additional ultraviolet light sources are configured in signal strength and choice of emitted ultraviolet wavelengths to act as another germicide for the cosmetic product contained in the container itself in addition to cooperating with one or more ultraviolet light sources emitting ultraviolet light wavelengths from the applicator wand to act as a germicide for the cosmetic product on the applicator wand itself.

9. The apparatus of claim 1, where the applicator wand has both
i) an outer body with an opening at a bottom of the outer body to the cosmetic product in the container, where the outer body is attached to a suction head to function as a syringe and dropper for a fluid-based cosmetic product, and
ii) a glass-based tubular inner portion that is optically coupled to the ultraviolet light source as the illumination source of the ultraviolet light wavelengths to emit the ultraviolet light wavelengths from within the glass-based tubular inner portion of the applicator wand.

10. The apparatus of claim 1, where the electronic control circuit includes a timer, where the control circuit with the timer is configured to ensure the cosmetic product housed within the container is exposed to the ultraviolet light wavelengths for a fixed amount of time during a sanitizing session, where the electronic control circuit is configured to use one or more sensors to detect when the cap is secured onto the container before activating the one or more ultraviolet light sources to emit the ultraviolet light wavelengths merely when the applicator wand is inside the container, which is indicated by the cap being secured onto the container.

11. A method for a sanitizer for cosmetic product, comprising:
manufacturing an applicator wand for the cosmetic product that is optically coupled to an ultraviolet light source as an illumination source of ultraviolet light wavelengths;
configuring the applicator wand to provide the source of the ultraviolet light wavelengths to be a germicide for bacteria in the cosmetic product on the applicator wand, where the ultraviolet light source is configured to emit the ultraviolet light wavelengths in a range extending between 202-225 nm, where the applicator wand is configured to be used with a container to contain the cosmetic product, where a cap of the container is configured to be attached to a body of the container; and
configuring the ultraviolet light source and an electronic control circuit to be powered by one or more batteries electrically connected to the ultraviolet light source and the electronic control circuit.

12. The method of claim 11, further comprising:
manufacturing one or more illumination lights that are electrically connected to the one or more batteries, and that are physically attached to i) the cap, ii) the applicator wand, iii) the container, and iv) any combination of these, where the one or more illumination lights are configured to emit light in a range of wavelengths within a visible spectrum, and where the one or more illumination lights are configured to illuminate an area where the cosmetic product is intended to be applied.

13. The method of claim 12, where the cap houses the electronic control circuit, which is configured to engage a mechanical locking mechanism between the cap and the body of the container for a duration of time the ultraviolet light source is actively emitting the ultraviolet light wavelengths.

14. The method of claim 11, further comprising:
manufacturing the cap with the one or more batteries located in and embedded inside the cap, where the batteries are rechargeable batteries and connect to a recharging circuit which includes any of i) a USB port, ii) USB-C port, iii) micro-USB, and iv) mini-USB port, formed in the cap in order to recharge the rechargeable batteries.

15. The method of claim 11, where the applicator wand is connected into the cap, where the applicator wand is a translucent structure made partially or entirely from glass, where the one or more ultraviolet light sources are located in the cap and optically couple to a body and head of the applicator wand.

16. The method of claim 11, further comprising:
manufacturing where the applicator wand with a head that connects to a body of the applicator wand, where the applicator head is configured to be removable and replaceable from the body of the applicator wand.

17. The method of claim 11, where the one or more ultraviolet light sources are built into the cap and then optically coupled to the applicator wand; and then, two or more additional ultraviolet light sources are built into walls of the container where the two or more additional ultraviolet light sources are arranged in a pattern inside the container, and where the one or more ultraviolet light sources are configured to emit the ultraviolet light at 222 nm.

18. The method of claim 17, where the container has the one or more additional ultraviolet light sources built into an interior of the container, where the container has i) a tubular or ii) rectangular tube-shape, where the one or more additional ultraviolet light sources are configured in signal strength and choice of emitted ultraviolet wavelengths to act as another germicide for the cosmetic product contained in the container itself in addition to cooperating with one or more ultraviolet light sources emitting the ultraviolet light wavelengths from the applicator wand to act as the germicide for the cosmetic product on the applicator wand itself.

19. The method of claim 11, further comprising:
manufacturing the applicator wand to have both
i) an outer body with an opening at a bottom the outer body to the cosmetic product in the container, where the outer body is attached to a suction head to function as a syringe and dropper for a fluid-based cosmetic product, and
ii) a glass-based tubular inner portion that is optically coupled to the ultraviolet light source as the illumination source of the ultraviolet light wavelengths to emit the ultraviolet light wavelengths from within the glass-based tubular inner portion of the applicator wand.

20. The method of claim 11, further comprising:
manufacturing the cap to contain the electronic control circuit, which includes a timer, where the control circuit with the timer is configured to ensure the cosmetic product housed within the container is exposed to the ultraviolet light wavelengths for a fixed amount of time during a sanitizing session, where the electronic control circuit is configured to use one or more sensors to detect when the cap is secured onto the container before activating the one or more ultraviolet light sources to emit the ultraviolet light wavelengths merely when the applicator wand is inside the container.
